(19) United States
(12) Patent Application Publication
(10) Pub. No.: US 2026/0144770 A1
(43) Pub. Date: May 28, 2026

(54) COMPOSITION FOR INHIBITING SEBUM SECRETION CONTAINING A THYMOL ESTER-BASED COMPOUND

(71) Applicant: AMOREPACIFIC CORPORATION, Seoul (KR)

(72) Inventors: Hyesun SHIN, Yongin-si (KR); Suna KIM, Yongin-si (KR); Hyoung June KIM, Yongin-si (KR); Hyunjung CHOI, Yongin-si (KR)

(73) Assignee: AMOREPACIFIC CORPORATION, Seoul (KR)

(21) Appl. No.: 19/177,888

(22) Filed: Apr. 14, 2025

(30) Foreign Application Priority Data
Apr. 19, 2024 (KR) 10-2024-0052697

(51) Int. Cl. A61K 31/222; A61K 8/37; A61P 17/08; A61Q 5/00; A61Q 19/00

(52) U.S. Cl. CPC A61K 31/222; A61K 8/37; A61P 17/08; A61Q 5/008; A61Q 19/008

(57) ABSTRACT

A composition for inhibiting sebum secretion is disclosed. The composition contains 3,4,5-trimethoxycinnamate thymol ester, a stereoisomer, pharmaceutically acceptable salt, hydrate, or solvate thereof as an active ingredient. The composition can inhibit sebum secretion in the skin or scalp, and further has useful effects such as the prevention or improvement of seborrheic dermatitis, seborrheic scalp dermatitis, or acne, or the tightening of pores. Methods for inhibiting sebum secretion employing 3,4,5-trimethoxycinnamate thymol ester, a stereoisomer, pharmaceutically acceptable salt, hydrate, or solvate are disclosed.

### CROSS REFERENCE TO RELATED APPLICATION

The present application claims priority to Korean Patent Application No. 10-2024-0052697, filed Apr. 19, 2024, the entire contents of which are hereby incorporated by this reference.

### BACKGROUND

### Field

Disclosed herein is a composition for inhibiting sebum secretion, including 3,4,5-trimethoxycinnamate thymol ester or a stereoisomer, pharmaceutically acceptable salt, hydrate, or solvate thereof as an active ingredient, and methods for inhibiting sebum secretion, employing 3,4,5-trimethoxycinnamate thymol ester or a stereoisomer, pharmaceutically acceptable salt, hydrate, or solvate thereof.

### Description of the Related Art

Sebum secreted from the sebaceous glands plays important roles, such as providing the skin with moisture and luster, and protecting it from external microorganisms. However, excessive sebum secretion can also cause various skin diseases such as acne and seborrheic scalp dermatitis.

To solve this problem, many studies have been conducted and various substances have been developed as sebum-inhibiting materials, but to date, no substance that satisfies all needs has been found.

### SUMMARY

### Technical Problem

In one aspect of the present disclosure, it is intended to provide a composition capable of inhibiting or preventing sebum-related diseases such as seborrheic dermatitis by inhibiting sebum secretion of the skin or scalp.

### Technical Solution

In one aspect, the present disclosure provides a composition for inhibiting sebum secretion, comprising 3,4,5-trimethoxycinnamate thymol ester, a stereoisomer, pharmaceutically acceptable salt, hydrate, or solvate thereof as an active ingredient.

### Advantageous Effects

In one aspect, the composition according to the present disclosure can inhibit sebum secretion in the skin or scalp.

In one aspect, the composition according to the present disclosure can care for oily skin or oily scalp.

In one aspect, the composition according to the present disclosure can prevent or improve seborrheic dermatitis, seborrheic scalp dermatitis, or acne.

In one aspect, the composition according to the present disclosure can promote the tightening of pores in the skin.

### BRIEF DESCRIPTION OF THE DRAWINGS

FIGS. 1 and 2 show the results of observing the sebum secretion-inhibiting effects of the composition according to one embodiment of the present disclosure.

### DETAILED DESCRIPTION

Hereinafter, the present disclosure will be described in more detail by way of the following embodiments. However, the following embodiments are provided only for illustrative purposes to aid understanding of the present disclosure, and the scope and range of the present disclosure are not limited thereto.

In exemplary embodiments of the present disclosure, there is provided a composition for inhibiting sebum secretion, comprising 3,4,5-trimethoxycinnamate thymol ester, a stereoisomer, pharmaceutically acceptable salt, hydrate, or solvate thereof as an active ingredient.

In another exemplary embodiment of the present disclosure, there is provided a method for inhibiting sebum secretion, the method comprising administering a composition including 3,4,5-trimethoxycinnamate thymol ester, a stereoisomer, pharmaceutically acceptable salt, hydrate, or solvate thereof as an active ingredient.

In yet another exemplary embodiment of the present disclosure, there is provided a use of 3,4,5-trimethoxycinnamate thymol ester, a stereoisomer, pharmaceutically acceptable salt, hydrate, or solvate thereof for preparing a composition for inhibiting sebum secretion.

In still another exemplary embodiment of the present disclosure, there is provided a non-therapeutic use of 3,4,5-trimethoxycinnamate thymol ester, a stereoisomer, pharmaceutically acceptable salt, hydrate, or solvate thereof for inhibiting sebum secretion.

In still another exemplary embodiment of the present disclosure, there is provided 3,4,5-trimethoxycinnamate thymol ester, a stereoisomer, pharmaceutically acceptable salt, hydrate, or solvate thereof for inhibiting sebum secretion.

In one embodiment, the 3,4,5-trimethoxycinnamate thymol ester is (5-methyl-2-propan-2-ylphenyl) (E)-3-(3,4,5-trimethoxyphenyl)prop-2-enoate, which may be represented by Chemical Formula 1 below:

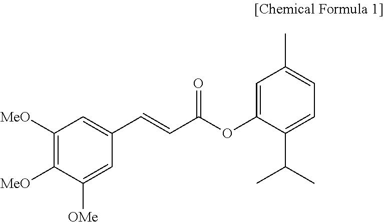 [SMILES: COc1cc(/C=C/C(=O)Oc2cc(C)ccc2C(C)C)cc(OC)c1OC]

As used herein, the term “isomer” includes in particular optical isomers (e.g., essentially pure enantiomers, essentially pure diastereomers or mixtures thereof), as well as conformation isomers (i.e., isomers which differ only in the angles of one or more chemical bonds), position isomers (in particular, tautomers) or geometric isomers (e.g., cis-trans isomers).

As used herein, the term “essentially pure”, for example, when used in connection with an enantiomer or diastereomer, means that a specific compound, for example an enantiomer or diastereomer, is present in an amount of at least about 90%, preferably at least about 95%, more preferably at least about 97% or at least about 98%, even more preferably at least about 99%, and even more preferably at least about 99.5% (w/w).

As used herein, the term “pharmaceutically acceptable” means that it can be or has been approved by the government or equivalent regulatory body as being suitable for use in animals, and more particularly, in humans, by avoiding significant toxic effects when used in conventional medicinal dosages, or is listed in a pharmacopoeia or recognized in other general pharmacopoeias.

As used herein, the term “pharmaceutically acceptable salt” means a salt according to one aspect of the present disclosure that is pharmaceutically acceptable and has the desired pharmacological activity of the parent compound. The salt may include (1) an acid addition salt formed of inorganic acid such as hydrochloric acid, hydrobromic acid, sulfuric acid, nitric acid, phosphoric acid, etc.; or formed of organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo [2,2,2]-oct-2-ene-1-carboxylic acid, glucoheptonic acid, 3-phenylpropionic acid, trimethylacetic acid, tert-butylacetic acid, lauryl sulfate, gluconic acid, glutamic acid, hydroxynaphthoic acid, salicylic acid, stearic acid, muconic acid; or (2) a salt formed when an acidic proton present in the parent compound is substituted.

As used herein, the term “hydrate” refers to a compound to which water is bound, and is a broad concept that includes an inclusion compound with no chemical bonding force between water and the compound.

As used herein, the term “solvate” refers to a high-order compound formed between a molecule or ion of a solute and a molecule or ion of a solvent.

In one embodiment, the composition may be for preventing or improving seborrheic dermatitis, seborrheic scalp dermatitis, folliculitis, acne, or seborrheic eczema.

In one embodiment, the composition may be for caring for oily skin or an oily scalp.

In one embodiment, the composition may be for tightening pores.

In one embodiment, the composition can inhibit sebum secretion induced by linoleic acid.

In one embodiment, the composition can reduce sebum secretion induced by linoleic acid by 40% or more.

For example, the composition can reduce sebum secretion induced by linoleic acid by 40% or more, 45% or more, 50% or more, 55% or more, or 60% or more, or 99% or less.

In one embodiment, the concentration of the active ingredient may be 0.01 to 5 wt % based on the total weight of the composition.

For example, the concentration of the active ingredient may be 0.01 wt % or more, 0.05 wt % or more, 0.1 wt % or more, 0.5 wt % or more, 1 wt % or more, 2 wt % or more, 3 wt % or more, or 4 wt % or more, and may be 5 wt % or less, 4 wt % or less, 3 wt % or less, 2 wt % or less, 1 wt % or less, 0.5 wt % or less, 0.1 wt % or less, or 0.05 wt % or less, based on the total weight of the composition.

In one embodiment, the composition may be a skin external application composition.

In one embodiment, the composition may be a non-therapeutic transdermal composition.

The skin external application or transdermal composition is a generic term that may include anything that is applied externally to the skin, and may include various types of cosmetics, etc.

In one embodiment, the composition may be a cosmetic composition.

The external form of the cosmetic composition contains a cosmetically or dermatologically acceptable medium or base. It may be provided in any formulation suitable for topical application, for example, in the form of a solution, a gel, a solid, a pasty anhydrous product, an emulsion obtained by dispersing an oil phase in an aqueous phase, a suspension, a microemulsion, a microcapsule, a microgranule, or ionic (liposome) and nonionic vesicular dispersion, or in the form of a cream, a toner, a lotion, a powder, an ointment, a spray, or a concealer stick. These compositions can be prepared according to conventional methods in the art. The composition according to the present disclosure may also be used in the form of a foam or in the form of an aerosol composition further containing a compressed propellant.

The cosmetic composition according to one embodiment of the present disclosure is not particularly limited in its formulation, and may be formulated into cosmetics such as a softening toner, an astringent toner, a nourishing toner, a nourishing cream, a massage cream, an essence, an eye cream, an eye essence, a cleansing cream, a cleansing foam, a cleansing water, a facial mask, a powder, a body lotion, a body cream, a body oil, and a body essence.

When the formulation of the cosmetic composition of the present disclosure is a paste, a cream, or a gel, animal fiber, plant fiber, wax, paraffin, starch, tragacanth, a cellulose derivative, polyethylene glycol, silicone, bentonite, silica, talc, zinc oxide, or the like may be used as a carrier component.

When the formulation of the cosmetic composition of the present disclosure is a powder or spray, lactose, talc, silica, aluminum hydroxide, calcium silicate or polyamide powder may be used as a carrier component, and in particular, in the case of a spray, a propellant such as chlorofluorohydrocarbon, propane/butane or dimethyl ether may be additionally included.

When the formulation of the cosmetic composition of the present disclosure is a solution or an emulsion, a solvent, a solvating agent or an emulsifying agent is used as a carrier component, and the examples include water, ethanol, isopropanol, ethyl carbonate, ethyl acetate, benzyl alcohol, benzyl benzoate, propylene glycol, 1,3-butyl glycol oil, glycerol aliphatic ester, polyethylene glycol or a fatty acid ester of sorbitan.

When the formulation of the cosmetic composition of the present disclosure is a suspension, a liquid diluent such as water, ethanol or propylene glycol, a suspending agent such as ethoxylated isostearyl alcohol, polyoxyethylene sorbitol ester and polyoxyethylene sorbitan ester, microcrystalline cellulose, aluminum metahydroxide, bentonite, agar, tragacanth, or the like may be used as a carrier component.

When the formulation of the cosmetic composition of the present disclosure is a surfactant-containing cleansing, aliphatic alcohol sulfate, aliphatic alcohol ether sulfate, sulfosuccinic acid monoester, isethionate, imidazolinium derivative, methyl taurate, sarcosinate, fatty acid amide ether sulfate, alkylamidobetaine, aliphatic alcohol, fatty acid glyceride, fatty acid diethanolamide, vegetable oil, linolin derivative, ethoxylated glycerol fatty acid ester, etc. may be used as a carrier ingredient.

The cosmetic composition of the present disclosure may further include a functional additive and a component included in a general cosmetic composition in addition to the above-described active ingredients. The functional additive may include a component selected from the group consisting of water-soluble vitamins, oil-soluble vitamins, polymer peptides, polymer polysaccharides, sphingo lipids, and seaweed extract.

In the cosmetic composition of the present disclosure, in addition to the above functional additive, a component included in a general cosmetic composition may be blended as necessary. In addition, other blending components, such as oil and fat ingredients, moisturizers, emollients, surfactants, organic and inorganic pigments, organic powders, ultraviolet absorbers, preservatives, disinfectants, antioxidants, plant extracts, pH adjusters, alcohols, pigments, fragrances, blood circulation promoters, cooling agents, antiperspirant, purified water, etc., may be additionally included.

In one embodiment, the composition may be a food composition.

In one embodiment, the composition may be a non-therapeutic oral composition.

The food composition or non-therapeutic oral composition according to the present disclosure may be a liquid or solid formulation, and may be a tablet, a capsule, a soft capsule, a pills, a granule, a beverage (drinking agent), a diet bar, a chocolate, a caramel formulation, or a confectionery formulation, but the formulation is not particularly limited.

In addition to the above active ingredients, the food composition or non-therapeutic oral composition according to the present disclosure may further include excipients, sugars, flavors, pigments, fats, proteins, etc., as needed.

In one embodiment, the 3,4,5-trimethoxycinnamate thymol ester, the stereoisomer, pharmaceutically acceptable salt, hydrate, or solvate thereof may be applied or ingested at an application or ingestion amount of 0.01 to 10 mg/kg/day.

In one embodiment, the composition may be a pharmaceutical composition.

The pharmaceutical composition according to one embodiment of the present disclosure may additionally contain pharmaceutical adjuvants such as preservatives, stabilizers, hydrating agents or emulsifying agents, salts for osmotic pressure control, and/or buffers, and other therapeutically useful substances, and may be formulated into various oral or parenteral dosage forms according to conventional methods.

The oral dosage forms include, for example, tablets, pills, hard and soft capsules, solutions, suspensions, emulsions, syrups, dusts, powders, fine granules, granules, pellets, etc., and these formulations may contain, in addition to the active ingredient, a surfactant, a diluent (e.g., lactose, dextrose, sucrose, mannitol, sorbitol, cellulose, and glycine), and a lubricant (e.g., silica, talc, stearic acid and its magnesium or calcium salts, and polyethylene glycol). The tablet may also contain a binder such as magnesium aluminum silicate, starch paste, gelatin, tragacanth, methylcellulose, sodium carboxymethylcellulose, and polyvinylpyrrolidone, and may optionally contain pharmaceutical additives such as a disintegrant, such as starch, agar, alginic acid or a sodium salt thereof, an absorbent, a coloring agent, a flavoring agent, and a sweetener. The tablet may be manufactured by a conventional mixing, granulating, or coating method.

In addition, the parenteral dosage form may be a transdermal dosage formulation, for example, a formulation such as an injection, a drop, an ointment, a lotion, a gel, a cream, a spray, a suspension, an emulsion, a suppository, a patch, etc., but is not limited thereto.

In one embodiment, a daily application amount of the active ingredient may be 0.01 to 10 mg/kg for the 3,4,5-trimethoxycinnamate thymol ester, the stereoisomer, pharmaceutically acceptable salt, hydrate, or solvate thereof.

Hereinafter, the present disclosure will be described in more detail by way of the following examples. However, the following examples are provided only for illustrative purposes to aid understanding of the present disclosure, and the scope and range of the present disclosure are not limited thereto.

### EXAMPLES

### Preparation of 3,4,5-trimethoxycinnamate thymol ester

Thymol trimethoxycinnamate thymol ester with CAS number 504394-57-4 (Melasolv) was obtained from COSMANN Co., Ltd. (Hwaseong-si, Gyeonggi-do, Korea).

### Experimental Example

### 1. Test of Sebum Secretion-Inhibiting Effect Using Human Sebocytes

Human sebocytes were cultured for 24 hours in an environment of 37° C. and 5% CO₂ with Sebocyte Complete Culture Media (Celprogen), and then treated with 100 μM linoleic acid and 0.1, 0.5, 1, and 2 ppm Melasolv and cultured for another 48 hours.

Thereafter, the cells were fixed with formaldehyde, and lipids were stained with 2 μM Bodipy. Fluorescence intensity was measured at a specific wavelength (Excitation/Emission: 485/528 nm) using a Fluorescent Plate Reader.

The data showed results as a percentage compared to the control group (100%) in which the sample was not treated.

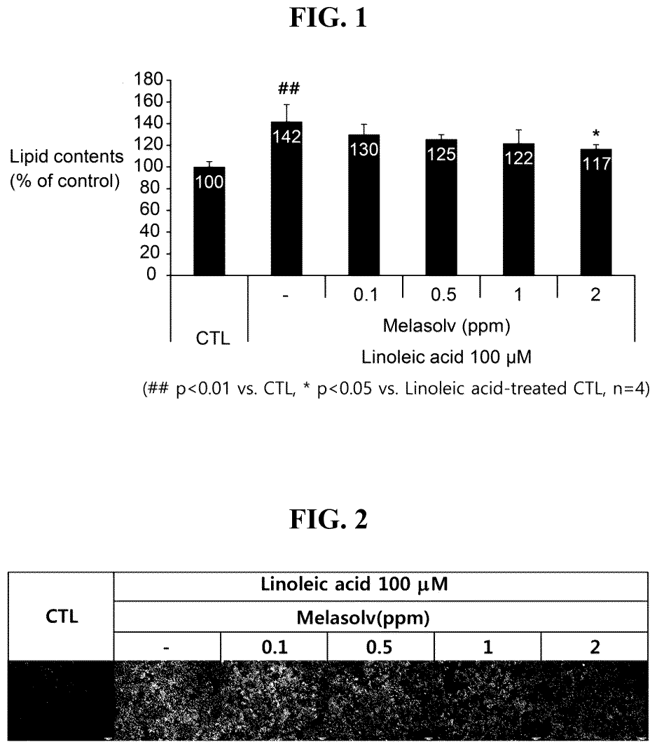

Referring to FIG. 1 (test of sebum secretion-inhibiting effect in vitro), as a result of treating the sebocytes with 100 μM linoleic acid, it was observed that sebum production was significantly increased (142%). Based on the amount of sebum production induced by linoleic acid, when treated with the inducer and Melasolv together, the amount of sebum production decreased in proportion to the concentration when treated with 0.1, 0.5, 1, and 2 ppm Melasolv, and Melasolv showed an effect of inhibiting sebum production by approximately 60.3% at a maximum of 2 ppm. Through these results, it was confirmed that Melasolv has the ability to inhibit sebum.

The fluorescence intensity results in FIG. 1 can be confirmed with the naked eye through the fluorescence photograph of FIG. 2, and it could be confirmed that sebum production was most inhibited at 2 ppm of Melasolv.

What is claimed is:

1. A method for inhibiting sebum secretion, the method comprising administering to a subject in need thereof an effective amount of a composition including 3,4,5-trimethoxycinnamate thymol ester, a stereoisomer, pharmaceutically acceptable salt, hydrate, or solvate thereof as an active ingredient.

2. The method according to claim 1, wherein the composition is for preventing or improving seborrheic dermatitis, seborrheic scalp dermatitis, folliculitis, acne, or seborrheic eczema.

3. The method according to claim 1, wherein the composition is for caring for oily skin or an oily scalp.

4. The method according to claim 1, wherein the composition is for tightening pores.

5. The method according to claim 1, wherein the concentration of the active ingredient is 0.01 to 5 wt % based on the total weight of the composition.

6. The method according to claim 1, wherein the composition is a skin external application composition.

7. The method according to claim 1, wherein the composition is a cosmetic composition.

8. The method according to claim 1, wherein the composition is a pharmaceutical composition.

9. The method according to claim 1, wherein a daily application amount of the active ingredient is 0.01 to 10 mg/kg.
